(19) United States
(12) Patent Application Publication
(10) Pub. No.: US 2020/0246390 A1
(43) Pub. Date: Aug. 6, 2020

(54) MULTIPOTENT ADULT PROJENITOR CELLS FOR TREATMENT OF ICH

(71) Applicant: ABT Holding Company, Cleveland, OH (US)

(72) Inventor: Robert William MAYS, Shaker Heights, OH (US)

(73) Assignee: ABT Holding Company, Cleveland, OH (US)

(21) Appl. No.: 16/265,373

(22) Filed: Feb. 1, 2019

(51) Int. Cl. A61K 35/36; A61K 9/00; A61K 35/28; A61P 7/04

(52) U.S. Cl. CPC A61K 35/36; A61K 9/0019; A61K 45/06; A61P 7/04; A61K 35/28

(57) ABSTRACT

Aspects of inventions herein described relate to administration of multipotent adult progenitor cells for treating intracerebral hemorrhage.

### FIELD OF THE INVENTION

The invention relates to intracerebral hemorrhage and to multipotent stem cells.

### GOVERNMENT FUNDING

No government funds were used in making the inventions herein disclosed.

### RELATED APPLICATIONS

This application does not claim priority of any previous applications.

### I

Intracerebral hemorrhage, (“ICH”) refers to any bleeding within the intracranial vault, including the brain parenchyma and surrounding meningeal space. ICH is a devastating disease affecting substantial populations in the US and worldwide. (See, for instance, Caceres and Goldstein (2012): Emerg Med Clin North Am 30(3) 771-794.) The incidence of spontaneous ICH worldwide is 24.6 per 100,000 person-years and there are approximately 40,000 to 67,000 cases per year in the United States. The 30-day mortality rate ranges from 35% to 52%. Approximately half of overall mortality occurs within the first 24 hours. Only 20% of survivors have full functional recovery at 6 months. It is thought that early and effective treatment is critically important. (See, for instance, van Asch et al. (2010): The Lancet Neurology 9(2) 167-176; Aguilar and Freeman (2010): Semin Neurol 30(5) 555-64, Broderick et al. (2007): Stroke 38(6) 2001-2023; Elliott and Smith (2010): Anesthesia & Analgesia 110(5) 1419-1427; and Feigin et al. (2009): The Lancet Neurology 8(4) 355-369.)

Intraparenchymal bleeds often arise from penetrating head trauma. They can also arise from depressed skull fractures. Additional causes include rupture of an aneurysm, arteriovenous malformation (AVM), bleeding within a tumor and acceleration-deceleration trauma. In patients over the age of 55, amyloid angiopathy is a frequent cause of intracerebral hemorrhage. Cerebral venous sinus thrombosis accounts for a very small fraction of ICH.

Primary ICH often is a manifestation of underlying small vessel disease. First, longstanding hypertension leads to hypertensive vasculopathy causing microscopic degenerative changes in the walls of small-to-medium penetrating vessels (lipohyalinosis). Second, cerebral amyloid angiopathy develops, characterized by the deposition of amyloid-beta peptide (AP3) in the walls of small leptomeningeal and cortical vessels. The mechanism leading to accumulation of amyloid is unknown; but, the consequences are well documented: degenerative changes in the vessel wall characterized by the loss of smooth muscle cells, wall thickening, luminal narrowing, microaneurysm formation and microhemorrhages. (See, for instance, Fisher, C M. (1971): J Neuropathol Exp Neurol 30(3) 536-50; Vinters H. (1987): Stroke 18(2) 311-324; and Viswanathan et al. (2011): Annals of Neurology 70(6) 871-880.)

Following initial vessel rupture, the hematoma causes direct mechanical injury to the brain parenchyma. Perihematomal edema develops within the first 3 hours from symptom onset and peaks between 10 to 20 days. Next, blood and plasma products mediate secondary injury processes including an inflammatory response, activation of the coagulation cascade, and iron deposition from hemoglobin degradation. Finally, a hematoma can continue to expand in up to 38 percent of patients during the first 24 hours. (See, for instance, Aronowski and Zhao (2011): Stroke 42(6): 781-6 and Brott et al. (1997): Stroke 28(1) 1-5.)

In sum, ICH is a type of brain injury that results from leakage of blood into the brain and accumulation of blood in the brain parenchyma. It can result from the rupture of an aneurysm, damage (such as perforation) of a cerebral artery or from an arteriovenous malformation (AVM). It is a devastating neurological injury, and it accounts for about 20% of all stroke-related injuries globally, and almost 30% in Japan and Asia. It has the highest mortality and the worst long-term outcomes of all stroke-related injuries and ICH is responsible for almost 50% of all stroke related deaths. Since hematoma volume is an independent determinant of ICH patient outcomes, early clot resolution is a primary clinical goal. However, there are, no FDA-approved therapies that improve ICH outcome. Emergency surgery to remove the clot (if possible) and rehabilitation are the only current standards of care; but, surgical management has limited utility and rehabilitation is a matter of coping with the damage rather than preventing or repairing it. Indeed, the most recent guidance from the AHA/ASA and the only recommended treatment for patients with an ICH is surgical removal of the clot—if surgery can be performed. There is no other recommended therapeutic intervention. (See, for instance, Hemphill III, J. C. et al. (2015): AHA/ASA Guideline, Stoke 46: 2032-2060.)

Clearly, there is an enormous need for ways of addressing ICH injury to reduce and to repair resulting brain injury. It is therefore among the objects of embodiments of the inventions herein disclosed to provide means and methods to improve outcomes in cases of ICH.

Heretofore, stem cells were not thought to be useful to treat ICH. ICH pathophysiology, as mentioned above, is thought to be driven by the presence and subsequent breakdown of red blood cells in the brain, releasing hemoglobin and its neurotoxic heme breakdown products. The extravasation of erythrocytes into the brain produces a space-occupying hematoma/clot that is associated with mechanical tissue destruction, edema formation, elevated intracranial pressure, enhanced microvascular compression, decreased blood flow and poor outcomes, which do not seem prone to remediation by cellular therapies.

As described herein, it has been found surprisingly that administration of multipotent adult stem cells as described herein to treat ICH has an unexpected and surprisingly effective therapeutically beneficial on ICH outcomes, as shown by measures of hematoma volume, cerebral blood flow/perfusion result and functional neurological assessments.

### II

A few of the many embodiments encompassed by the present description are summarized in the following numbered paragraphs. The numbered paragraphs are self-referential. In particular, the phase “in accordance with any of the foregoing or the following” used in these paragraphs refers to the other paragraphs. The phrase means, in the following paragraphs, embodiments herein disclosed include both the subject matter described in the individual paragraphs taken alone and the subject matter described by the paragraphs taken in combination. In this regard, it is explicitly applicant's purpose in setting forth the following paragraphs to describe various aspects and embodiments particularly by the paragraphs taken alone or in any combination. That is, the paragraphs are a compact way of setting out and providing explicit written description of all the embodiments encompassed by them individually and in any combination with one another. Applicant specifically reserves the right at any time to claim any subject matter set out in any of the following paragraphs, alone or together with any other subject matter of any one or more of the other paragraphs, including any combination of any values therein set forth, taken alone or in any combination with any other value therein set forth. Should it be required, applicant specifically reserves the right to set forth all of the combinations herein set forth in full in this application or in any successor applications having benefit of this application.

p1. A method of treating intracerebral hemorrhage in a subject, comprising administering to a subject in need thereof multipotent adult progenitor cells that are not embryonic stem cells, not embryonic germ cells, and not germ cells, can differentiate into at least one cell type of each of at least two of the endodermal, ectodermal, and mesodermal embryonic lineages, and are allogeneic or xenogeneic to the subject.

p2. A method of treating intracerebral hemorrhage in a subject, comprising administering to a subject in need thereof multipotent adult progenitor cells that are not embryonic stem cells, not embryonic germ cells, and not germ cells, express telomerase and are allogeneic or xenogeneic to the subject.

p3. A method of treating intracerebral hemorrhage in a subject, comprising administering to a subject in need thereof multipotent adult progenitor cells that are not embryonic stem cells, not embryonic germ cells, and not germ cells, are positive for oct4 and are allogeneic or xenogeneic to the subject.

p4. A method of treating intracerebral hemorrhage in a subject, comprising administering to a subject in need thereof multipotent adult progenitor cells that are not embryonic stem cells, not embryonic germ cells, and not germ cells, undergone at least 40 cell doublings in culture prior to their use, and are allogeneic or xenogeneic to the subject.

p5. A method according to any of the foregoing and/or the following, wherein can differentiate into at least one cell type of each of at least two of the endodermal, ectodermal, and mesodermal embryonic lineages.

p6. A method according to any of the foregoing or the following, wherein said cells express telomerase.

p7. A method according to any of the foregoing or the following, wherein said cells are positive for oct4.

p8. A method use according to any of the foregoing or the following, wherein said cells express telomerase and are positive for oct4.

p9. A method according to any of the foregoing or the following, wherein said cells express telomerase and have undergone at least 40 cell doublings prior to their use.

p10. A method according to any of the foregoing or the following, wherein said cells express oct4 and have undergone at least 40 cell doublings prior to their use.

p11. A method according to any of the foregoing or the following, wherein said cells express telomerase, are positive for oct4 and have undergone at least 40 cell doublings prior to their use.

p12. A method according to any of the foregoing or the following, wherein said cells express any one or more of rex-1, rox-1, or sox-2.

p13. A method according to any one of any of the foregoing or the following, wherein said cells have a normal karyotype.

p14. A method according to any one of any of the foregoing or the following, wherein said cells are not tumorigenic.

p15. A method according to any of the foregoing or the following, wherein said cells do not form teratomas.

p16. A method according to any of the foregoing or the following, wherein said cells are not genetically altered.

p17. A method according to any of the foregoing or the following, wherein said cells are genetically altered.

p18. A method according to any of the foregoing or the following, wherein said cells are not immunogenic in said subject.

p19. A method according to any of the foregoing or the following, wherein said cells can differentiate into at least one cell type of each of the endodermal, ectodermal, and mesodermal embryonic lineages.

p20. A method according to any of the foregoing or the following, wherein said cells are mammalian cells.

p21. A method according to any of the foregoing or the following, wherein said cells are human cells.

p22. A method according to any of the foregoing or the following, wherein said cells are derived from cells isolated from any one of placental tissue, umbilical cord tissue, umbilical cord blood, bone marrow, blood, spleen tissue, thymus tissue, spinal cord tissue, adipose tissue, and liver tissue.

p23. A method according to any of the foregoing or the following, wherein said cells are derived from bone marrow.

p24. A method according to any of the foregoing or the following, wherein the subject is a human.

p25. A method according to any of the foregoing or the following, wherein one or more doses of 10⁴ to 10⁸ of said cells per kilogram of the subject's mass are used.

p26. A method according to any of the foregoing or the following, wherein one or more doses of 10⁶ to 5×10⁷ of said cells per kilogram of the subject's mass are used.

p27. A method according to any of the foregoing or the following, wherein in addition to said cells, one or more growth factors, differentiation factors, signaling factors, and/or factors that increase homing are used concurrently.

p28. A method according to any of the foregoing or the following, wherein an antimicrobial agent, an anti-fungal agent, an anti-viral agent or a combination thereof is used concurrently.

p29. A method according to any of the foregoing or the following, wherein said cells are in a formulation comprising one or more other pharmaceutically active agents.

p30. A method according to any of the foregoing or the following, wherein said cells are administered parenterally.

p32. A method according to any of the foregoing or the following, wherein said cells are administered intravenously.

p33. A method according to any of the foregoing or the following, wherein said cells are administered stereotactically.

p34. A method according to any of the foregoing or the following, wherein said cells are administered any one or more of 1, 5, 10, 15, 30, 45 or 60 minutes following ICH, or 60, 90, 120, 150 or 180 minutes following ICH, or 1, 2, 3, 4, 5, 6, 7, 8, 9, 10, 11 or 12 hours following ICH, or 12, 18, 24, 30, 36 or 40 hours after ICH, or 1, 2, 3, 4, 5, 6 or 7 days following ICH, or 1, 2, 3, 4, 5, 6, 7 or 8 weeks following ICH, or any combination of the foregoing and/or any later time.

### III

“A” or “an” as used herein means: one and more than one; at least one. Where the plural form is used herein, it generally includes the singular.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created in which the cells have been selected for enhanced potency to achieve the effects described in this application. Following release from storage, and prior to administration, it may be preferable to again assay the cells for potency. This can be done using any of the assays, direct or indirect, described in this application or otherwise known in the art. Then cells having the desired potency can then be administered. Banks can be made using autologous cells (derived from the organ donor or recipient). Or banks can contain cells for allogeneic uses.

“Co-administer” as used herein means to administer in conjunction with one another, together, coordinately, including simultaneous or sequential administration of two or more agents.

“Comprising” as used herein means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of” as used herein is a synonym of “comprising” (see above).

“Conditioned cell culture medium” is a term well-known in the art and refers to medium in which cells have been grown. As used herein the phrase means that cells are grown in the medium for a sufficient time to secrete factors that are effective for cell growth of a specified type for which the medium is being conditioned.

“Decrease” and “decreasing” and similar terms are used herein generally to mean to lessen in amount or value or effect, as by comparison to another amount, value or effect. For instance, decreasing the severity of ICH can mean decreasing hematoma volume and/or decreasing functional impairment, such as by comparison, respectively to the previous volume or functional impairment due to an ICH.

“Effective amount” as used herein generally means an amount which provides the desired local or systemic effect. For example, an effective amount is an amount sufficient to effectuate a beneficial or desired clinical result. For instance, an effective amount for treating ICH is an amount that decreases hematoma volume and/or improves and/or increases cerebral circulation and/or perfusion; and/or decreases neurological and/or functional impairment and/or improves function, such as motor function, balance and the like.

The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

“Effective route” as used herein generally means a route which provides for delivery of an agent to a desired compartment, system, or location. For example, an effective route is one through which an agent can be administered to provide at the desired site of action an amount of the agent sufficient to effectuate a beneficial or desired clinical result.

“ICH” as used herein is an acronym for and has the same meaning as “Intracerebral hemorrhage”.

“Includes” and “including” as used herein are not limiting and mean much the same as “comprises” and “comprising”.

“Increase” and “increasing” as used herein means to make greater in size, amount, intensity, or degree, such as a biological event or property, including to induce (as from zero or an inactive state). For instance, an effective amount for treating ICH is, for instance, an amount that increases cerebral circulation and/or perfusion; and/or improves function, such as motor function, balance and the like, such as by comparison to previous post-ICH function.

“Intracerebral hemorrhage” (“ICH”) is intracranial bleeding into brain tissue, the brain ventricles, or both. Causes ICH include but are not limited to bleeding from or as a result of any one or more of aneurysms, arteriovenous malformations, brain tumors and brain trauma. An ICH is also referred to as a “brain bleed.”

The term “isolated” as used herein refers to a cell or cells which are not associated with one or more cells or one or more cellular components that are associated with the cell or cells in vivo. An “enriched population” means a relative increase in numbers of a desired cell relative to one or more other cell types in vivo or in primary culture.

However, as used herein, the term “isolated” does not indicate the presence of only stem cells. Rather, the term “isolated” indicates that the cells are removed from their natural tissue environment and are present at a higher concentration as compared to the normal tissue environment. Accordingly, an “isolated” cell population may further include cell types in addition to stem cells and may include additional tissue components. This also can be expressed in terms of cell doublings, for example. A cell may have undergone 10, 20, 30, 40 or more doublings in vitro or ex vivo so that it is enriched compared to its original numbers in vivo or in its original tissue environment (e.g., bone marrow, peripheral blood, adipose tissue, etc.).

“MAPC” is an acronym for “multipotent adult progenitor cell.” It refers to a cell that is not an embryonic stem cell or germ cell. MAPC can be characterized in a number of alternative descriptions, each of which conferred novelty to the cells when they were discovered. They can, therefore, be characterized by one or more of those descriptions. First, they have extended replicative capacity in culture without being genetically engineered, transformed (tumorigenic) and with a normal karyotype. This means that these cells express telomerase (i.e., have telomerase activity). Second, they can give rise to cell progeny of more than one germ layer, such as two or all three germ layers (i.e., endoderm, mesoderm and ectoderm) upon differentiation. Third, although they are not embryonic stem cells or germ cells, they can express markers of these primitive cell types so that MAPCs can express one or more of oct4, rex-1, and rox-1. They can also express sox-2. Rex-1 is controlled by oct4, which activates downstream expression of rex-1. Rox-1 and sox-2 are expressed in non-ES cells. Accordingly, the cell type that is designated “MAPC” may be characterized by alternative basic characteristics that describe the cell via some of its novel properties.

The term “adult” in MAPC is non-restrictive. It refers to a non-embryonic somatic cell, such as, post-natal. MAPCs do not form teratomas in vivo. This acronym was first used in U.S. Pat. No. 7,015,037 to describe a pluripotent cell isolated from bone marrow. However, cells with pluripotential markers and/or differentiation potential have been discovered subsequently and, for purposes of this invention, may be equivalent to those cells first designated “MAPC.” Essential descriptions of the MAPC type of cell are provided in the Summary of the Invention above.

MAPC represents a more primitive progenitor cell population than MSC (Verfaillie, C. M., Trends Cell Biol 12:502-8 (2002); Jahagirdar, B. N., et al., Exp Hematol, 29:543-56 (2001); Reyes, M. and C. M. Verfaillie, Ann N YAcad Sci, 938:231-233 (2001); Jiang, Y. et al., Exp Hematol, 30896-904 (2002); and Jiang, Y. et al., Nature, 418:41-9. (2002)).

MAPC may not be immunogenic. MAPC may be immunosuppressive. MAPC may or may not be genetically altered to improve their characteristics. MAPC may or may not be used with or without adjunctive immunosuppressive treatment. Additional aspects of MAPC are described herein.

“May” as used herein the word “may” means the same as “optionally” and even where it is not stated, as used herein, “may” includes also that it “may not”. That is, a statement that something may be, means as well that it also may not be. That is, as used herein, “may” includes “may not”, explicitly, and applicant reserves the right to claim subject matter accordance therewith. For instance, as used herein, the statement that MAPC may be administered with other agents, also means that MAPC may be administered without any other agents. For a another example, as used herein the statement that MAPC may be genetically engineered also means that MAPC may be not genetically engineered.

“Multipotent”, with respect to MAPC as used herein refers to the ability of MAPC to give rise to cell lineages of more than more than one of the three primitive germ layers: endoderm, mesoderm and ectoderm upon differentiation, such as two or all three.

“MultiStem®” is the trade name for a cell preparation based on the MAPCs of U.S. Pat. No. 7,015,037, i.e., a non-embryonic stem, non-germ cell as described above. MultiStem® is prepared according to cell culture methods disclosed in this patent application, particularly, lower oxygen and higher serum. MultiStem® is highly expandable, karyotypically normal, and does not form teratomas in vivo. It can differentiate into cell lineages of more than one germ layer and can express one or more of telomerase, oct4, rex-1, rox-1, and sox-2.

“Oct-4” is a member of the POU family of transcription factors (1). The mouse protein was first identified and classified as Oct-3. The human homologue was initially classified as Oct-3 based on its 87% homology to the mouse Oct-3 protein at the amino acid level. Subsequently, two human Oct-3 transcripts have been identified (Takeda, et al. Nucleic Acids Research 20(17) 4613-20 (1992), Oct-3A and 3B, which are alternative splice products of the same gene. Oct-3A has been identified and renamed Oct-4 by some groups, and Oct-3/4 by others. Still other groups have divided oct 4 into oct 4A and oct4B as alternative transcripts. The A transcript (i.e., oct 4, oct3A, oct3/4), giving rise to a nuclear protein, has been associated with pluripotency and is confined to part of exon 1. The B transcript is found in numerous cell types as a cytoplasmic protein and covers the remainder of exon 1 as well as exons 2-5.

“Optionally” as used herein means much the same as “may”. The statement that X optionally includes A as used herein includes both X includes A and X does not include A.

“Pharmaceutically acceptable carrier” is any pharmaceutically acceptable medium for the cells used in the present invention. Such a medium may retain isotonicity, cell metabolism, pH, and the like. It is compatible with administration to a subject in vivo, and can be used, therefore, for cell delivery and treatment.

“Progenitor cells” are cells produced during differentiation of a stem cell that have some, but not all, of the characteristics of their terminally-differentiated progeny. Defined progenitor cells, such as “cardiac progenitor cells,” are committed to a lineage, but not to a specific or terminally differentiated cell type. The term “progenitor” as used in the acronym “MAPC” does not limit these cells to a particular lineage.

The term “reduce” as used herein means to prevent as well as decrease. In the context of treatment, to “reduce” is to both prevent or ameliorate one or more clinical symptoms. A clinical symptom is one (or more) that has or will have, if left untreated, a negative impact on the quality of life (health) of the subject.

“Selecting” a cell with a desired level of potency can mean identifying (as by assay), isolating, and expanding a cell. This could create a population that has a higher potency than the parent cell population from which the cell was isolated. The “parent” cell population refers to the parent cells from which the selected cells divided. “Parent” refers to an actual P1→F1 relationship (i.e., a progeny cell). So if cell X is isolated from a mixed population of cells X and Y, in which X is an expressor and Y is not, one would not classify a mere isolate of X as having enhanced expression. But, if a progeny cell of X is a higher expressor, one would classify the progeny cell as having enhanced expression.

“Self-renewal” as used herein refers to the ability to produce replicate daughter stem cells having differentiation potential that is identical to those from which they arose. A similar term used in this context is “proliferation.”

“Stem cell” as used herein means a cell that can undergo self-renewal (i.e., progeny with the same differentiation potential) and also produce progeny cells that are more restricted in differentiation potential.

“Subject” as used herein means a vertebrate, such as a mammal, such as a human. Mammals include, but are not limited to, humans, dogs, cats, horses, cows, and pigs.

The term “therapeutically effective amount” as used herein refers to an amount determined to produce any beneficial therapeutic response in a subject. For example, effective amounts of therapeutic cells or cell-associated agents may prolong the survivability of the patient and/or inhibit overt clinical symptoms. Treatments that are therapeutically effective within the meaning of the term as used herein, include treatments that improve a subject's quality of life even if they do not improve the disease outcome per se. For instance, therapeutically effective can mean reducing the volume of a hemorrhage, improving cerebral blood flow, and/or improving neurological and/or behavior function, such as following an ICH. Such therapeutically effective amounts are readily ascertained by one of ordinary skill in the art.

“Treat,” “treating,” or “treatment” are used broadly in relation to the invention and each such term means in essence administration of cells as described herein, in particularly advantageous embodiments with the beneficial effect of one or more but not necessarily any or all of preventing, ameliorating, inhibiting, or curing a deficiency, dysfunction, disease, or other deleterious process, including those that interfere with and/or result from a therapy. For instance, treating can mean reducing the volume of a hemorrhage, improving cerebral blood flow, and/or improving neurological and/or behavior function, such as following an ICH. Such aspects of treatment are readily ascertained by one of ordinary skill in the art.

“Validate” as used herein means to confirm. In the context of the invention, one confirms that a cell is an expressor with a desired potency. This is so that one can then use that cell (in treatment, banking, drug screening, etc.) with a reasonable expectation of efficacy. Accordingly, to validate means to confirm that the cells, having been originally found to have/established as having the desired activity, in fact, retain that activity. Thus, validation is a verification event in a two-event process involving the original determination and the follow-up determination. The second event is referred to herein as “validation.”

### III

### Brief Description of the Drawings

Various features and advantages of the embodiments herein described can be fully appreciated as the same becomes better understood when considered in light of the accompanying drawings:

FIG. 1A and FIG. 1B: MULTISTEM® CELLS REDUCE HEMATOMA VOLUME FOLLOWING COLLAGENASE-INDUCED ICH

Placebo (PBS, n=10) or MultiStem® (n=11) were intravenously administered to mice at 2 hour post-collagenase induced ICH. Hematoma volume was assessed by MRI (T2W) using a 7T small animal MRI. Representative coronal brain images for day 3 and day 7 are provided in

FIG. 1A shows a dramatic benefit of MultiStem® cells on hematoma volume.

FIG. 1B depicts data from all mice over a 21 day assessment period.

Data are presented as mean+/−SEM and were analyzed by Student's t-test within each time point. **p<0.01 vs. placebo treated ICH mice.

Details are provided in Example 3.

FIG. 2A and FIG. 2B: MULTISTEM® CELLS IMPROVE CEREBRAL PERFUSION FOLLOWING COLLAGENASE-INDUCED ICH

Placebo (PBS, n=10) or MultiStem® (n=11) were intravenously administered to mice at 2 hour post-collagenase induced ICH. Cerebral perfusion was assessed by MRI (ASL; FAIR-RARE) using a 7T small animal MRI.

FIG. 2A—Representative coronal brain images.

FIG. 2B—Quantified perfusions data.

Data indicate that MultiStem® improves cerebral perfusion over the first week after ICH. Data are presented as mean+/−SEM and were analyzed by Student's t-test within each time point. * p<0.05, ** p<0.01 vs. placebo treated ICH mice.

Details are provided in Example 4.

FIG. 3A, FIG. 3B and FIG. 3C: MULTISTEM® CELLS IMPROVE MOTOR FUNCTION FOLLOWING COLLAGENASE INDUCED ICH

Placebo (PBS, n=10) or MultiStem® (n=l1) were intravenously administered to mice at 2 hour post-collagenase induced ICH. Neurological assessment of motor function was assessed at day 7 post-injury (or in sham-operated mice; n=8).

FIG. 3A—Grip Strength Test results.

FIG. 3B—Narrow Beam Task test results.

FIG. 3C—Elevated Body Swing Task test results.

Data are mean+/−SEM and were compared using a One Way ANOVA followed by Tukey's post-hoc test. * p<0.05, **p<0.01, *** p<0.001, ns=not significant.

Details are provided in Example 5.

### V

As described herein aspects of the invention relate to the administration of MAPC (as herein defined) to a subject who has experienced an intracerebral hemorrhagic bleed, otherwise known as a hemorrhagic stroke. These patients have no therapeutic interventions, other than surgical evacuation of the blood clot, if size and location of the clot in the brain are amenable to surgery.

Aspects of the invention as herein described provide methods of administering the cells to subject suffering from and/or in need of treatment for intracerebral hemorrhage, so as to have the beneficial effect of one or more but not necessarily any or all of preventing, ameliorating, inhibiting, or curing an intracerebral hemorrhage. Cells and methods in accordance therewith are described below.

Embodiments of the invention provides administration MultiStem® cells via an intravenous route after ICH onset, for instance in the subacute time frame (hours). Administration can by a variety of routes and times as may be found to be effective.

Without being limited to any particular mechanism of action, it noted that MultiStem® cells modulate acute inflammatory responses in other preclinical and clinical injuries. It may be that action of MultiStem® in treating ICH is mediated in some aspect by similar action of MultiStem® on acute inflammatory response occurring after ICH.

The cells can naturally achieve these effects (i.e., not genetically or pharmaceutically modified). However, the cells also can be genetically or pharmaceutically modified to increase effectiveness and/or improve their properties.

In one embodiment, the cells have undergone a desired number of cell doublings in culture. For example, the cells have undergone at least 10-40 cell doublings in culture, such as 30-35 cell doublings, and wherein the cells are not transformed and have a normal karyotype. If cells are transformed or tumorigenic, and it is desirable to use them for infusion, such cells may be disabled so they cannot form tumors in vivo, as by treatment that prevents cell proliferation into tumors. Such treatments are well known in the art.

Oct4, which otherwise is specific to ES, EG, and germ cells, is considered to be a marker of undifferentiated cells that have broad differentiation abilities. Oct4 also is generally thought to have a role in maintaining a cell in an undifferentiated state. Oct4 belongs to the POU (Pit Oct Unc) family of transcription factors and is a DNA binding protein that is able to activate the transcription of genes, containing an octameric sequence called “the octamer motif” within the promoter or enhancer region. Oct4 is expressed at the moment of the cleavage stage of the fertilized zygote until the egg cylinder is formed. The function of Oct4 is to repress differentiation inducing genes (i.e., FoxaD3, hCG) and to activate genes promoting pluripotency (FGF4, Utfl, Rexl). Sox2, a member of the high mobility group (HMG) box transcription factors, cooperates with Oct4 to activate transcription of genes expressed in the inner cell mass. It is essential that Oct4 expression in embryonic stem cells is maintained between certain levels. Overexpression or downregulation of >50% of Oct4 expression level will alter embryonic stem cell fate, with the formation of primitive endoderm/mesoderm or trophectoderm, respectively. In vivo, Oct4 deficient embryos develop to the blastocyst stage, but the inner cell mass cells are not pluripotent. Instead they differentiate along the extraembryonic trophoblast lineage.

Sall4, a mammalian Spalt transcription factor, is an upstream regulator of Oct4, and is therefore important to maintain appropriate levels of Oct4 during early phases of embryology. When Sall4 levels fall below a certain threshold, trophectodermal cells will expand ectopically into the inner cell mass.

The cells include, but are not limited to, characteristics in the following numbered embodiments:

pb1. Isolated expanded non-embryonic stem, non-germ cells, the cells having undergone at least 10-40 cell doublings in culture, wherein the cells express oct4, are not transformed, and have a normal karyotype.

pb2. The non-embryonic stem, non-germ cells of 1 above that further express one or more of telomerase, rex-1, rox-1, or sox-2.

pb3. The non-embryonic stem, non-germ cells of 1 above that can differentiate into at least one cell type of at least two of the endodermal, ectodermal, and mesodermal embryonic lineages.

pb4. The non-embryonic stem, non-germ cells of 3 above that further express one or more of telomerase, rex-1, rox-1, or sox-2.

pb5. The non-embryonic stem, non-germ cells of 3 above that can differentiate into at least one cell type of each of the endodermal, ectodermal, and mesodermal embryonic lineages.

pb6. The non-embryonic stem, non-germ cells of 5 above that further express one or more of telomerase, rex-1, rox-1, or sox-2.

pb7. Isolated expanded non-embryonic stem, non-germ cells that are obtained by culture of non-embryonic, non-germ tissue, the cells having undergone at least 40 cell doublings in culture, wherein the cells are not transformed and have a normal karyotype.

pb8. The non-embryonic stem, non-germ cells of 7 above that express one or more of oct4, telomerase, rex-1, rox-1, or sox-2.

pb9. The non-embryonic stem, non-germ cells of 7 above that can differentiate into at least one cell type of at least two of the endodermal, ectodermal, and mesodermal embryonic lineages.

pb10. The non-embryonic stem, non-germ cells of 9 above that express one or more of oct4, telomerase, rex-1, rox-1, or sox-2.

pb11. The non-embryonic stem, non-germ cells of 9 above that can differentiate into at least one cell type of each of the endodermal, ectodermal, and mesodermal embryonic lineages.

pb12. The non-embryonic stem, non-germ cells of 11 above that express one or more of oct4, telomerase, rex-1, rox-1, or sox-2.

pb13. Isolated expanded non-embryonic stem, non-germ cells, the cells having undergone at least 10-40 cell doublings in culture, wherein the cells express telomerase, are not transformed, and have a normal karyotype.

pb14. The non-embryonic stem, non-germ cells of 13 above that further express one or more of oct4, rex-1, rox-1, or sox-2.

pb15. The non-embryonic stem, non-germ cells of 13 above that can differentiate into at least one cell type of at least two of the endodermal, ectodermal, and mesodermal embryonic lineages.

pb16. The non-embryonic stem, non-germ cells of 15 above that further express one or more of oct4, rex-1, rox-1, or sox-2.

pb17. The non-embryonic stem, non-germ cells of 15 above that can differentiate into at least one cell type of each of the endodermal, ectodermal, and mesodermal embryonic lineages.

pb18. The non-embryonic stem, non-germ cells of 17 above that further express one or more of oct4, rex-1, rox-1, or sox-2.

pb19. Isolated expanded non-embryonic stem, non-germ cells that can differentiate into at least one cell type of at least two of the endodermal, ectodermal, and mesodermal embryonic lineages, said cells having undergone at least 10-40 cell doublings in culture.

pb20. The non-embryonic stem, non-germ cells of 19 above that express one or more of oct4, telomerase, rex-1, rox-1, or sox-2.

pb21. The non-embryonic stem, non-germ cells of 19 above that can differentiate into at least one cell type of each of the endodermal, ectodermal, and mesodermal embryonic lineages.

pb22. The non-embryonic stem, non-germ cells of 21 above that express one or more of oct4, telomerase, rex-1, rox-1, or sox-2.

Selecting Cells

The MAPC can be used as isolated and expanded as described herein. MAPC also can be selected for particular properties before use, with or without using genetic engineering techniques.

Selecting a cell with a desired level of potency can mean identifying (as by assay), isolating, and expanding a cell. This could create a population that has a higher potency than the parent cell population from which the cell was isolated. The “parent” cell population refers to the parent cells from which the selected cells divided. “Parent” refers to an actual P1→F1 relationship (i.e., a progeny cell). So if cell X is isolated from a mixed population of cells X and Y, in which X is an expressor and Y is not, one would not classify a mere isolate of X as having enhanced expression. But, if a progeny cell of X is a higher expressor, one would classify the progeny cell as having enhanced expression.

To select a cell that achieves the desired effect would include both an assay to determine if the cells achieve the desired effect and would also include obtaining those cells. The cell may naturally achieve the desired effect in that the effect is not achieved by an exogenous transgene/DNA. But an effective cell may be improved by being incubated with or exposed to an agent that increases the effect. The cell population from which the effective cell is selected may not be known to have the potency prior to conducting the assay. The cell may not be known to achieve the desired effect prior to conducting the assay. As an effect could depend on gene expression and/or secretion, one could also select on the basis of one or more of the genes that cause the effect.

Selection could be from cells in a tissue. For example, in this case, cells would be isolated from a desired tissue, expanded in culture, selected for achieving the desired effect, and the selected cells further expanded.

Selection could also be from cells ex vivo, such as cells in culture. In this case, one or more of the cells in culture would be assayed for achieving the desired effect and the cells obtained that achieve the desired effect could be further expanded.

Cells could also be selected for enhanced ability to achieve the desired effect. In this case, the cell population from which the enhanced cell is obtained already has the desired effect. Enhanced effect means a higher average amount per cell than in the parent population.

The parent population from which the enhanced cell is selected may be substantially homogeneous (the same cell type). One way to obtain such an enhanced cell from this population is to create single cells or cell pools and assay those cells or cell pools to obtain clones that naturally have the enhanced (greater) effect (as opposed to treating the cells with a modulator that induces or increases the effect) and then expanding those cells that are naturally enhanced.

However, cells may be treated with one or more agents that will induce or increase the effect. Thus, substantially homogeneous populations may be treated to enhance the effect.

If the population is not substantially homogeneous, then, it is preferable that the parental cell population to be treated contains at least 100 of the desired cell type in which enhanced effect is sought, more preferably at least 1,000 of the cells, and still more preferably, at least 10,000 of the cells. Following treatment, this sub-population can be recovered from the heterogeneous population by known cell selection techniques and further expanded if desired.

Thus, desired levels of effect may be those that are higher than the levels in a given preceding population. For example, cells that are put into primary culture from a tissue and expanded and isolated by culture conditions that are not specifically designed to produce the effect may provide a parent population. Such a parent population can be treated to enhance the average effect per cell or screened for a cell or cells within the population that express greater degrees of effect without deliberate treatment. Such cells can be expanded then to provide a population with a higher (desired) expression.

Use and Administration

In some embodiments the cells are used as the sole active agent of therapy. In some embodiments of the invention MAPCs are used together with one or more other agents and/or therapeutic modalities as the primary therapeutic modality. In some embodiments of the invention the cells are used as an adjunctive therapeutic modality, that is, as an adjunct to another, primary therapeutic modality. In some embodiments the cells are used as the sole active agent of an adjunctive therapeutic modality. In others the cells are used as an adjunctive therapeutic modality together with one or more other agents or therapeutic modalities. In some embodiments the cells are used both as primary and as adjunctive therapeutic agents and/or modalities. In both regards, the cells can be used alone in the primary and/or in the adjunctive modality. They also can be used together with other therapeutic agents or modalities, in the primary or in the adjunctive modality or both.

As discussed above, a primary treatment, such as a therapeutic agent, therapy, and/or therapeutic modality, targets (that is, is intended to act on) the primary dysfunction, such as a disease, that is to be treated. An adjunctive treatment, such as a therapy and/or a therapeutic modality, can be administered in combination with a primary treatment, such as a therapeutic agent, therapy, and/or therapeutic modality, to act on the primary dysfunction, such as a disease, and supplement the effect of the primary treatment, thereby increasing the overall efficacy of the treatment regimen. An adjunctive treatment, such as an agent, therapy, and/or therapeutic modality, also can be administered to act on complications and/or side effects of a primary dysfunction, such as a disease, and/or those caused by a treatment, such as a therapeutic agent, therapy, and/or therapeutic modality. In regard to any of these uses, one, two, three, or more primary treatments may be used together with one, two, three, or more adjunctive treatments.

In some embodiments MAPCs are administered to a subject prior to onset of ICH. In embodiments the cells are administered while the ICH and/or resulting dysfunction is developing. In some embodiments the cells are administered after the ICH and or resulting dysfunction has been established. MAPCs can be administered at any stage in the development, persistence, and/or propagation of the ICH or related dysfunction or after it recedes.

Cells can be administered any one or more of 1, 5, 10, 15, 30, 45 or 60 minutes before or following ICH, or 60, 90, 120, 150 or 180 minutes before or following ICH, or 1, 2, 3, 4, 5, 6, 7, 8, 9, 10, 11 or 12 hours before of following ICH, or 12, 18, 24, 30, 36 or 40 hours after ICH, or 1, 2, 3, 4, 5, 6 or 7 days following ICH, or 1, 2, 3, 4, 5, 6, 7 or 8 weeks following ICH, or any combination of the foregoing and/or any later time.

Cells can be administered immediately to 60 minutes after ICH, 30 to 90 minutes after ICH, 1 to 6 hours after ICH, 5 to15 hours after ICH, 10 to 20 hours after ICH, 15 to 25 hours after ICH, 20 to 40 hours after ICH, 1 to 5 days after ICH, 1 day to 1 week after ICH, 1 to 2 weeks after ICH, 1 to several weeks after ICH, several weeks to 1 month after ICH, 1 or several months after ICH, any time after ICH.

Cells also can administered before ICH, at any time in advance of ICH or at any of the times or intervals before ICH noted in foregoing two paragraphs.

As discussed above, embodiments of the invention provide cells and methods for primary or adjunctive therapy. In certain embodiments of the invention, the cells are administered to an allogeneic subject (i.e., are allogeneic to the subject). In some embodiments they are autologous to the subject. In some embodiments they are syngeneic to the subject. In some embodiments the cells are xenogeneic to a subject. Whether allogeneic, autologous, syngeneic, or xenogeneic, in various embodiments of the invention the MAPCs are only weakly immunogenic or are non-immunogenic in the subject. In embodiments the MAPCs are sufficiently low immunogenicity non-immunogenic so that they do not provoke adverse immune responses in general when administered to allogeneic and/or xenogeneic subjects that they can be used as “universal” donor cells without tissue typing and matching.

Furthermore in this regard MAPCs in various embodiments can be administered without adjunctive immunosuppressive treatment. In accordance with various embodiments of the invention the MAPCs can also be stored and maintained in cell banks, and thus can be kept available for use when needed.

In all of these regards and others, embodiments of the invention provide MAPCs from mammals, including in one embodiment humans, and in other embodiments non-human primates, rats and mice, and dogs, pigs, goats, sheep, horses, and cows. MAPCs prepared from mammals as described above can be used in all of the methods and other aspects of the invention described herein.

MAPCs in accordance with various embodiments of the invention can be isolated from a variety of compartments and tissues of such mammals in which they are found, including but not limited to, bone marrow, peripheral blood, cord blood, blood, spleen, liver, muscle, brain, adipose tissue, placenta and others discussed below. MAPCs in some embodiments are cultured before use.

In some embodiments MAPC are isolated from bone marrow. In some particular embodiments in this regard, MAPC are isolated from human bone marrow.

In many embodiments MAPCs are not genetically engineered.

In some embodiments MAPC are genetically engineered. MAPC can be genetically engineered for a wide variety of purposes, such as those well known to the art. For instance, they can be engineered to have improved growth characteristics, to improve their therapeutic efficacy, to express one or more exogenous genes to produce beneficial substance, and to alter their immunological profiles.

In some embodiments genetically engineered MAPCs are produced by in vitro culture. In some embodiments genetically engineered MAPCs are produced from a transgenic organism.

Formulations

MAPCs can be prepared from a variety of tissues, such as bone marrow cells, as discussed in greater detail elsewhere herein.

In many embodiments the purity of MAPCs for administration to a subject is about 100%. In other embodiments it is 95% to 100%. In some embodiments it is 85% to 95%. Particularly in the case of admixtures with other cells, the percentage of MAPCs can be 2%-5%, 3%-7%, 5%-10%, 7%-15%, 10%-15%, 10%-20%, 15%-20%, 20%-25%, 25%-30%, 30%-35%, 35%-40%, 40%-45%, 45%-50%, 60%-70%, 70%-80%, 80%-90%, or 90%-95%.

In some embodiments the purity of the cells for administration is about 100% (substantially homogeneous). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the cells have undergone, for example, 10-20, 20-30, 30-40, 40-50 or more cell doublings.

Treatment of disorders or diseases or the like with MAPCs may be with undifferentiated MAPCs. Treatment also may be with MAPCs that have been treated so that they are committed to a differentiation pathway. Treatment also may involve MAPCs that have been treated to differentiate into a less potent stem cell with limited differentiation potential. It also may involve MAPCs that have been treated to differentiate into a terminally differentiated cell type. The best type or mixture of MAPCs will be determined by the particular circumstances of their use, and it will be a matter of routine design for those skilled in the art to determine an effective type or combination of MAPCs in this regard.

The choice of formulation for administering MAPCs for a given application will depend on a variety of factors. Prominent among these will be the species of subject, the nature of the intracerebral hemorrhage being treated and its state and distribution in the subject, the nature of other therapies and agents that are being administered, the optimum route for administration of the MAPCs, survivability of MAPCs via the route, the dosing regimen, and other factors that will be apparent to those skilled in the art. In particular, for instance, the choice of suitable carriers and other additives will depend on the exact route of administration and the nature of the particular dosage form.

Cell survival may be an important determinant of the efficacy of therapies using MAPCs. This is true for both primary and adjunctive therapies. Another concern arises when target sites are inhospitable to cell seeding and cell growth. This may impede access to the site and/or engraftment there of therapeutic MAPCs. In embodiments the invention comprises the use of measures to increase cell survival and/or to overcome problems posed by barriers to seeding and/or growth.

Various additives often will be included to enhance the stability, sterility, and isotonicity of the compositions, such as antimicrobial preservatives, antioxidants, chelating agents, and buffers, among others.

Prevention of the action of microorganisms can be ensured by various antibacterial and antifungal agents, for example.

A pharmaceutically acceptable preservative or cell stabilizer can be employed to increase the life of MAPC compositions.

If such additives are included, it is well within the purview of the skilled artisan to select compositions that will not affect the viability or efficacy of the MAPCs.

Standard texts, such as “REMINGTON'S PHARMACEUTICAL SCIENCE,” 17th edition, 1985, incorporated herein by reference, may be consulted to prepare suitable preparations, without undue experimentation.

Among preferred embodiments are solutions for injection, including those for local, I.V. infusion and stereotactic injection.

In some embodiments, MAPCs are formulated in a unit dosage injectable form.

The skilled artisan can readily determine the amount of cells and optional additives, vehicles, and/or carrier in compositions to be administered in methods of the invention.

Additional Active Agents

MAPCs may be administered with other pharmaceutically active agents. In some embodiments one or more of such agents are formulated together with MAPCs for administration. In some embodiments the MAPCs and the one or more agents are in separate formulations. In some embodiments the compositions comprising the MAPCs and/or the one or more agents are formulated with regard to adjunctive use with one another.

MAPCs may be administered in a formulation comprising immunosuppressive agents, such as any combination of any number of a corticosteroid, cyclosporin A, a cyclosporin-like immunosuppressive agent, cyclophosphamide, anti-thymocyte globulin, azathioprine, FK-506, and a macrolide-like immunosuppressive agent.

Such agents also include antibiotic agents, antifungal agents, and antiviral agents, to name just a few other pharmacologically active substances and compositions that may be used in accordance with embodiments of the invention.

Typical antibiotics and anti-mycotic compounds include, but are not limited to, penicillin, streptomycin, amphotericin, ampicillin, gentamicin, kanamycin, mycophenolic acid, nalidixic acid, neomycin, nystatin, paromomycin, polymyxin, puromycin, rifampicin, spectinomycin, tetracycline, tylosin, zeocin, and cephalosporins, aminoglycosides, and echinocandins.

MAPC also may be administered in combination with agents that enhance homing of the cells to sites of injury; i.e., sites of injury resulting from ICH. For example, MAPC may be administered with growth factors and trophic signaling agents, such as cytokines, such as for example stromal cell derived factor-1 (SDF-1), stem cell factor (SCF), angiopoietin-1, placenta-derived growth factor (PIGF), granulocyte-colony stimulating factor (G-CSF), and those that stimulate expression of endothelial adhesion molecules.

They may be administered to a subject as a pre-treatment, along with MAPCs, or after MAPCs have been administered, to promote homing to desired sites and to achieve improved therapeutic effect, either by improved homing or by other mechanisms. Such factors may be combined with MAPCs in a formulation suitable for them to be administered together. Alternatively, such factors may be formulated and administered separately.

Order of administration, formulations, doses, frequency of dosing, and routes of administration of other active agents and MAPCs generally will vary with the ICH being treated, its severity, the subject, other therapies that are being administered, the stage of the disorder or disease, and prognostic factors, among others. General regimens that have been established for other treatments provide a framework for determining appropriate dosing in MAPC-mediated direct or adjunctive therapy. These, together with the additional information provided herein, will enable the skilled artisan to determine appropriate administration procedures in accordance with embodiments of the invention, without undue experimentation.

In embodiments cells are formulated suitably for treating brain injury, including the brain injuries and/or dysfunctions and/or disorders and/or diseases set forth herein. In embodiments, the formulations are effective for parenteral administration. In embodiments the formulations are effective for I.V. infusion. In embodiments the formulations are effective for stereotactic injection.

Routes of Administration

MAPCs can be administered to a subject by any of a variety of routes known to those skilled in the art that may be used to administer cells to a subject.

In various embodiments the MAPCs are administered to a subject by any route for effective delivery of cell therapeutics. In some embodiments the cells are administered by injection, including local and/or systemic injection. In certain embodiments the cells are administered within and/or in proximity to the site of the ICH they are intended to treat. In some embodiments, the cells are administered by injection at a location not in proximity to the site of the dysfunction. In some embodiments the cells are administered by systemic injection, such as intravenous injection.

Among methods that may be used in this regard in embodiments of the invention are methods for administering MAPCs by systemic injection. Systemic injection, such as intravenous injection, offers one of the simplest and least invasive routes for administering MAPCs. In some cases, these routes may require high MAPC doses for optimal effectiveness and/or homing by the MAPCs to the target sites. In a variety of embodiments MAPCs may be administered by targeted and/or localized injections to ensure optimum effect at the target sites.

MAPCs may be administered to the subject through a hypodermic needle by a syringe in some embodiments of the invention. In various embodiments, MAPCs are administered to the subject through a catheter. In a variety of embodiments, MAPCs are administered by surgical implantation. Further in this regard, in various embodiments of the invention, MAPCs are administered to the subject by implantation using an arthroscopic procedure. In some embodiments MAPCs are administered to the subject by stereotactic injection.

Dosing

Compositions can be administered in dosages and by techniques well known to those skilled in the medical and veterinary arts taking into consideration such factors as the age, sex, weight, and condition of the particular patient, and the formulation that will be administered (e.g., solid vs. liquid). Doses for humans or other mammals can be determined without undue experimentation by the skilled artisan, from this disclosure, the documents cited herein, and the knowledge in the art.

The dose of MAPCs appropriate to be used in accordance with various embodiments of the invention will depend on numerous factors. It may vary considerably for different circumstances. The parameters that will determine optimal doses of MAPCs to be administered for primary and adjunctive therapy generally will include some or all of the following: the ICH being treated and its stage; the species of the subject, their health, gender, age, weight, and metabolic rate; the subject's immunocompetence; other therapies being administered; and expected potential complications from the subject's history or genotype. The parameters may also include: whether the MAPCs are syngeneic, autologous, allogeneic, or xenogeneic; their potency (specific activity); the site and/or distribution that must be targeted for the MAPCs to be effective; and such characteristics of the site such as accessibility to MAPCs and/or engraftment of MAPCs. Additional parameters include co-administration with MAPCs of other factors (such as growth factors and cytokines). The optimal dose in a given situation also will take into consideration the way in which the cells are formulated, the way they are administered, and the degree to which the cells will be localized at the target sites following administration. Finally, the determination of optimal dosing necessarily will provide an effective dose that is neither below the threshold of maximal beneficial effect nor above the threshold where the deleterious effects associated with the dose of MAPCs outweighs the advantages of the increased dose.

The optimal dose of MAPCs for some embodiments will be in the range of doses used for autologous, mononuclear bone marrow transplantation. It can be estimated by extrapolation from animal studies taking into account differences in size (mass) and metabolic factors, and from dosage requirements established for other cell therapies, such as transplant therapies.

In embodiments optimal doses range from 10⁴ to 10⁹ MAPC cells/kg of recipient mass per administration. In embodiments optimal doses per administration will be between 10⁵ to 10⁸ MAPC cells/kg. In embodiments optimal dose per administration will be 5×10⁵ to 5.×10⁷ MAPC cells/kg. In embodiments optimal doses per administration will be any of 1, 2, 3, 4, 5, 6, 7, 8, or 9.times.10⁶ to any of 1, 2, 3, 4, 5, 6, 7, 8, or 9.times.10⁷.

By way of reference, some of the mid-high doses in the foregoing are analogous to the doses of nucleated cells used in autologous mononuclear bone marrow transplantation. Some of the mid-lower doses are analogous to the number of CD3+ cells/kg used in autologous mononuclear bone marrow transplantation.

It is to be appreciated that a single dose may be delivered all at once, fractionally, or continuously over a period of time. The entire dose also may be delivered to a single location or spread fractionally over several locations.

In various embodiments, MAPCs may be administered in an initial dose, and thereafter maintained by further administration of MAPCs. MAPCs may be administered by one method initially, and thereafter administered by the same method or one or more different methods. The subject's MAPC levels can be maintained by the ongoing administration of the cells. Various embodiments administer the MAPCs either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimen for initial administration and further doses or for sequential administrations may all be the same or may be variable. Appropriate regimens can be ascertained by the skilled artisan, from this disclosure, the documents cited herein, and the knowledge in the art.

MAPCs may be administered in many frequencies over a wide range of times, such as until a desired therapeutic effect is achieved. In some embodiments, MAPCs are administered over a period of less than one day. In other embodiment they are administered over two, three, four, five, or six days. In some embodiments MAPCs are administered one or more times per week, over a period of weeks. In other embodiments they are administered over a period of weeks for one to several months. In various embodiments they may be administered over a period of months. In others they may be administered over a period of one or more years. Generally lengths of treatment will be proportional to the length of the disease process, the effectiveness of the therapies being applied, and the condition and response of the subject being treated.

In some embodiments, MAPCs are administered one time, two times, three times, or more than three times until a desired therapeutic effect is achieved or administration no longer appears to be likely to provide a benefit to the subject. In some embodiments MAPCs are administered continuously for a period of time, such as by intravenous drip. Administration of MAPCs may be for a short period of time, for days, for weeks, for months, for years, or for longer periods of time.

In embodiments, a single bolus is administered. In embodiments two or more administrations of a single bolus are administered separated in time by one or more days. In embodiments each dose is administered by I.V. infusions over any period of time from several minutes to several hours. In embodiments a single dose of cells is administered by stereotactic injection. In embodiments, two or more doses are administered to the same or different areas of the brain by stereotactic injection. In embodiments involving bolus, IV, and stereotactic injection for treating brain injury in this regard, the dose of cells per administration is from 10⁴ to 10⁹ MAPC cells/kg of recipient mass per administration. In embodiments the dose is from 10⁵ to 10⁸ MAPC cells/kg. In embodiments the dose is from 5.×10⁵ to 5.×.10⁷ MAPC cells/kg. In embodiments the dose is 1, 2, 3, 4, 5, 6, 7, 8, or 9.×10⁶ to any of 1, 2, 3, 4, 5, 6, 7, 8, or 9.×10⁷.

Isolation and Growth of MAPCs

Methods of MAPC isolation are known in the art. See, for example, U.S. Pat. No. 7,015,037, and these methods, along with the characterization (phenotype) of MAPCs, are incorporated herein by reference. MAPCs can be isolated from multiple sources, including, but not limited to, bone marrow, placenta, umbilical cord and cord blood, muscle, brain, liver, spinal cord, blood or skin. It is, therefore, possible to obtain bone marrow aspirates, brain or liver biopsies, and other organs, and isolate the cells using positive or negative selection techniques available to those of skill in the art, relying upon the genes that are expressed (or not expressed) in these cells (e.g., by functional or morphological assays such as those disclosed in the above-referenced applications, which have been incorporated herein by reference).

MAPCs have also been obtained my modified methods described in Breyer et al., Experimental Hematology, 34:1596-1601 (2006) and Subramanian et al., Cellular Programming and Reprogramming: Methods and Protocols; S. Ding (ed.), Methods in Molecular Biology, 636:55-78 (2010), incorporated by reference for these methods.

Isolation and Growth of MAPCS as Described in U.S. Pat. No. 7,015,037

Methods of MAPC isolation are known in the art from, for instance, humans, rat, mouse, dog and pig. Illustrative methods are described in, for instance, U.S. Pat. No. 7,015,037 and PCT/US02/04652 (published as WO 02/064748), and these methods, along with a characterization of MAPCs disclosed therein, by way of illustration and non-limiting example only, are incorporated herein by reference.

MAPCs were initially isolated from bone marrow, and were subsequently established from other tissues, including brain and muscle (Jiang, Y. et al. (2002): Nature 418: 41-49). MAPCs can be isolated from many sources, including, but not limited to bone marrow, placenta, umbilical cord and cord blood, muscle, brain, liver, spinal cord, blood, adipose tissue and skin. For example, MAPCs can be derived from bone marrow aspirates, which can be obtained by standard means available to those of skill in the art (see, for example, Muschler, G. F. et al. (1997) J Bone Joint Surg Am; 79(11):1699-709 and Batinic, D. et al. (1990): Bone Marrow Transplant 6(2): 103-7.).

Human MAPC Phenotype Under Conditions Set Forth in U.S. Pat. No. 7,015,037

Immunophenotypic analysis by FACS of human MAPCs obtained after 22-25 cell doublings indicated that the cells do not express CD31, CD34, CD36, CD38, CD45, CD50, CD62E and -P, HLA-DR, Muc18, STRO-1, cKit, Tie/Tek; and express low levels of CD44, HLA-class I, and .beta.2-microglobulin, but express CD10, CD13, CD49b, CD49e, CDw90, Flk1 (N>10).

Once cells underwent >40 doublings in cultures re-seeded at about 2×10³/cm², the phenotype became more homogenous, and no cell expressed HLA class-I or CD44 (n=6). When cells were grown at higher confluence, they expressed high levels of Muc18, CD44, HLA class I, and .beta.2-microglobulin, which is similar to the phenotype described for MSC (N=8) (Pittenger, 1999).

Immunohistochemistry showed that human MAPCs grown at about 2×10³3/cm² seeding density expressed EGF-R, TGF-R1 and -2, BMP-R1A, PDGF-R1a and -B, and that a small subpopulation (between 1 and 10%) of MAPCs stained with anti-SSEA4 antibodies (Kannagi, R, 1983).

Using Clontech cDNA arrays the expressed gene profile of human MAPCs cultured at seeding densities of about 2×10³ cells/cm² for 22 and 26 cell doublings was determined:

A. MAPCs did not express CD31, CD36, CD62E, CD62P, CD44-H, cKit, Tie, receptors for IL1, IL3, IL6, IL11, G CSF, GM-CSF, Epo, Flt3-L, or CNTF, and low levels of HLA-class-I, CD44-E and Muc-18 mRNA.

B. MAPCs expressed mRNA for the cytokines BMP1, BMP5, VEGF, HGF, KGF, MCP1; the cytokine receptors Flk1, EGF-R, PDGF-R1.alpha., gp130, LIF-R, activin-R1 and -R2, TGFR-2, BMP-R1A; the adhesion receptors CD49c, CD49d, CD29; and CD10.

C. MAPCs expressed mRNA for hTRT and TRF1; the POU domain transcription factor oct-4, sox-2 (required with oct-4 to maintain undifferentiated state of ES/EC (Uwanogho, D. (1995): Mech Dev 49(1-2): 23-36); sox 11 (neural development), sox 9 (chondrogenesis) (Lefebvre V. et al. (1998): Matrix Biol 16(9): 529-40); homeodeomain transcription factors Hox-a4 and -a5 (cervical and thoracic skeleton specification; organogenesis of respiratory tracts) (Packer A I (2000): Dev Dyn 217(1): 62-74); Hox-a9 (myelopoiesis) (Lawrence H. (1997): Blood 89(6): 1922-30); Dlx4 (specification of forebrain and peripheral structures of head) (Akimenko M A (1994): J Neurosci (6): 3475-86), MSX1 (embryonic mesoderm, adult heart and muscle, chondro- and osteogenesis) (Foerst-Potts L. (1997) Dev Dyn 209(1): 70-84); PDX1 (pancreas) (Offield M F et al. (1996): Development. 122(3): 983-95).

D. Presence of oct-4, LIF-R, and hTRT mRNA was confirmed by RT-PCR.

E. In addition, RT-PCR showed that rex-1 mRNA and rox-1 mRNA were expressed in MAPCs.

Oct-4, rex-1 and rox-1 were expressed in MAPCs derived from human and murine marrow and from murine liver and brain. Human MAPCs expressed LIF-R and stained positive with SSEA-4. Finally, oct-4, LIF-R, rex-1 and rox-1 mRNA levels were found to increase in human MAPCs cultured beyond 30 cell doublings, which resulted in phenotypically more homogenous cells. In contrast, MAPCs cultured at high density lost expression of these markers. This was associated with senescence before 40 cell doublings and loss of differentiation to cells other than chondroblasts, osteoblasts, and adipocytes. Thus, the presence of oct-4, combined with rex-1, rox-1, and sox-2, correlated with the presence of the most primitive cells in MAPCs cultures.

Culturing MAPC

Methods for culturing MAPCs are well-known in the art. (See for instance, U.S. Pat. No. 7,015,037, which is herein incorporated by reference as to methods for culturing MAPCs.) The density for culturing MAPCs can vary from about 100 cells/cm² or about 150 cells/cm² to about 10,000 cells/cm², including about 200 cells/cm² to about 1500 cells/cm² to about 2000 cells/cm². The density can vary between species. Additionally, optimal density can vary depending on culture conditions and source of cells. It is within the skill of the ordinary artisan to determine the optimal density for a given set of culture conditions and cells.

Also, effective atmospheric oxygen concentrations of less than about 10%, including about 3 to 5%, can be used at any time during the isolation, growth, and differentiation of MAPCs in culture.

Additional Culture Methods

In additional experiments, the density at which MAPCs are cultured can vary from about 100 cells/cm² or about 150 cells/cm² to about 10,000 cells/cm², including about 200 cells/cm² to about 1500 cells/cm² to about 2000 cells/cm². The density can vary between species. Additionally, optimal density can vary depending on culture conditions and source of cells. It is within the skill of the ordinary artisan to determine the optimal density for a given set of culture conditions and cells.

Also, effective atmospheric oxygen concentrations of less than about 10%, including about 1-5% and, especially, 3-5%, can be used at any time during the isolation, growth and differentiation of MAPCs in culture.

Cells may be cultured under various serum concentrations, e.g., about 2-20%. Fetal bovine serum may be used. Higher serum may be used in combination with lower oxygen tensions, for example, about 15-20%. Cells need not be selected prior to adherence to culture dishes. For example, after a Ficoll gradient, cells can be directly plated, e.g., 250,000-500,000/cm². Adherent colonies can be picked, possibly pooled, and expanded.

In one embodiment, used in the experimental procedures in the Examples, high serum (around 15-20%) and low oxygen (around 3-5%) conditions were used for the cell culture. Specifically, adherent cells from colonies were plated and passaged at densities of about 1700-2300 cells/cm² in 18% serum and 3% oxygen (with PDGF and EGF).

### EXAMPLES

The following examples are provided by way of illustration only and are in no way limiting, exclusive or exhaustive of the many aspects and embodiments of inventions herein disclosed.

### Example 1—Preparation of MAPC

Human MultiStem® MAPC of Athersys Inc., Cleveland, were used in the Examples described below. These are human bone marrow derived MAPCs isolated from a bone marrow aspirate, obtained with consent from a healthy donor, and processed according to previously described methods, essentially as described in Penn, M S et al., Circ Res 2012; 110(2):304-11; Maziarz, R T et al., Biology of Blood and Marrow Transplantation 2012; 18(2 Sup):S264-S265; and clinicaltrials.gov # NCT01436487, # NCT01240915 and # NCT01841632). In brief, MAPCs were cultured in fibronectin-coated plastic tissue culture flasks under low oxygen tension in a humidified atmosphere of 5% CO₂. Cells were cultured in MAPC culture media (low-glucose DMEM [Life Technologies Invitrogen] supplemented with FBS (Atlas Biologicals, Fort Collins, Colo.), ITS liquid media supplement [Sigma], MCDB [Sigma], platelet-derived growth factor (R&D Systems, Minneapolis, Minn.), epidermal growth factor (R&D Systems), dexamethasone ([Sigma], penicillin/streptomycin [Life Technologies Invitrogen], 2-Phospho-L-ascorbic acid [Sigma, St. Louis, Mo.), and linoleic acid-albumin (Sigma). Cells were passaged every 3-4 d, harvested using trypsin/EDTA (Life Technologies Invitrogen, Carlsbad, Calif.). The cells were positive for CD49c and CD90 and negative for MHC class II and CD45. The cells were subsequently frozen at population doubling 30-35 in cryovials in the vapor phase of liquid nitrogen at a concentration of 1-10×10⁶ in 1 ml (PlasmaLyte, 5% HSA and 10% DMSO). Immediately prior to their use, MAPCs were thawed and then used directly.

### Example 2—Collagenase ICH Induction in Rats

A mouse collagenase model of ICH was used for the study as previously described (Sukumari-Ramesh et al., J Neurotrauma 29(18):2798-804 (2012). Briefly, adult male C57Bl/6J mice (8-10 weeks old) were placed into a stereotactic frame and a 0.5-mm diameter burr hole was drilled over the parietal cortex, 2.2 mm lateral to the bregma. A 26-G Hamilton syringe, loaded with 0.04 μI of bacterial type IV collagenase in 0.5 μI saline was lowered 3 mm deep from the skull surface directly into the left striatum. The syringe was depressed at a rate of 450 nl/min and left in place for several minutes after the procedure to prevent solution reflux and excess diffusion. After the syringe was removedbone wax was used to close the burr hole, the incision was surgically stapled, and mice were keptwarm until recovery of the righting reflex. For all studies, littermates were used to reduce a source of experimental variability.

Animals were randomized to receive either intravenous (IV) saline (control; n=10) or MultiStem® cells (n=11) post-injury.

IV administration of cells or saline was given 2 hours after the initiation of the hemorrhagic bleed (2 hours after collagenase injection). All cell treated animals received 1 million cells.

Hematoma volume and cerebral perfusion was assessed by magnetic resonance imaging (MRI) over three weeks post-injury, as described below.

Neurobehavioral outcomes, including the grip strength test, narrow beam test, and elevated body swing task, were assessed at day 7 post-injury, as described below.

### Example 3—MAPC Reduces Hematoma Volume after ICH

IV administration of MultiStem® cells significantly reduced hematoma volume as early as 1 day after injury. This effect persisted over the first week post-ICH. Consistent with accelerated hematoma resolution. Mice were anesthetized with isoflurane (3% for induction, 1.5% for maintenance in a 2:1 mixture of N2/02 and imaged using a horizontal 7 Tesla BioSpec MRI spectrometer (Bruker Instruments) equipped with a 12-cm self-shielded gradient set (45 gauss/cm max). Radio frequency pulses were applied using a standard transmit/receive volume coil (72-mm internal diameter) actively decoupled from the two-channel Bruker quadrature receiver coil positioned over the centerline of the animal skull. Stereotaxic ear bars were used to minimize movement during the imaging procedure. Mouse temperature was maintained at 37±0.5° C. using a pad heated by a recirculating water bath. After positioning using a tri-planar fast low angle shot sequence, MR studies were performed using T2′-weighted MRI scans. The following parameters were used to acquire MRI: T2* mapping sequence (2D gradient echo sequence with multiple echoes; TE⋅ 5, 10, 15, 20, 25, and 30 ms; TR⋅ 3,000 ms; FOV=32 mm; 1-mm slice thickness (IS slices]; 256×256 matrix; NEX=2). Acquired images were segmented volumetrically using ImageJ software and hematoma volumes were computed. T2*W images were further processed using Bruker software to yield susceptibility-weighted images (Sehgal et al., 2006), providing an alternative method of segmentation and quality control reference for clot volumes. Both hematoma and ventricular volume were determined by drawing irregular regions of interest (ROIs) on all MRI sections containing the lesions/ventricle and the summed values(area) were multiplied by the thickness of the slice to calculate the volume. The analysis was done using ImageJ software.

Cell treated animals showed a statistically significant decrease in hematoma volume (approximately a 4-fold decrease) within one day of treatment. The decrease was statistically significant in the cell treated animals vs. the saline treated animals for at least the first 7 days.

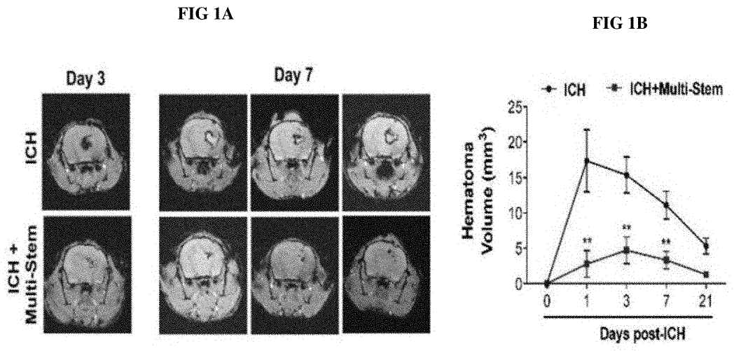

Results are shown in FIG. 1. Placebo (PBS, n=10) or MultiStem® (n=11) were intravenously administered to mice at 2 hour post-collagenase induced ICH. Hematoma volume was assessed by MRI (T2W) using a 7T small animal MRI. Representative coronal brain images for day 3 and day 7 are provided Panel A showing a dramatic benefic of MultiStem® on hematoma volume. Panel B depicts data from all mice over a 21 day assessment period. Data are presented as mean+/−SEM and were analyzed by Student's t-test within each time point. **p<0.01 vs. placebo treated ICH mice.

### Example 4—MAP C Improves Cerebral Perfusion after ICH

MultiStem® improved cerebral perfusion in and around the injured striatum for over 1-week post-ICH. Mice were anesthetized with isoflurane (3% for induction, 1.5% for maintenance in a 2:1 mixture of N2/O2 and imaged using a horizontal 7 Tesla BioSpec MRI spectrometer (Bruker Instruments) equipped with a 12-cm self-shielded gradient set (45 gauss/cm max). Radio frequency pulses were applied using a standard transmit/receive volume coil (72-mm internal diameter) actively decoupled from the two-channel Bruker quadrature receiver coil positioned over the centerline of the animal skull. Stereotaxic ear bars were used to minimize movement during the imaging procedure. Mouse temperature was maintained at 37±0.5° C. using a pad heated by a recirculating water bath. After positioning using a tri-planar fast low angle shot sequence, MR studies were performed using T2-weighted MRI scans. The following parameters were used to acquire MRI: T2-fluid attenuation inversion recovery sequence (RARE-IR, Tl ⋅ 2000; TR ⋅ 10,000 ms; TE ⋅ 36 ms; RARE factor=8; FOV=32 mm; 256×256 matrix; 1-mm slice thickness; JS slices. The analysis was done using ImageJ software.

Blood flow in cell treated animals was statistically significantly improved for the first 7 days after treatment when compared to saline treated injured animals. This illustrates that IV infusion of the MultiStem® cell product results in the acute improvement of blood flow in the brain following onset of the hemorrhagic stroke, likely resulting in less edema, tissue damage and disruption of neural circuitry.

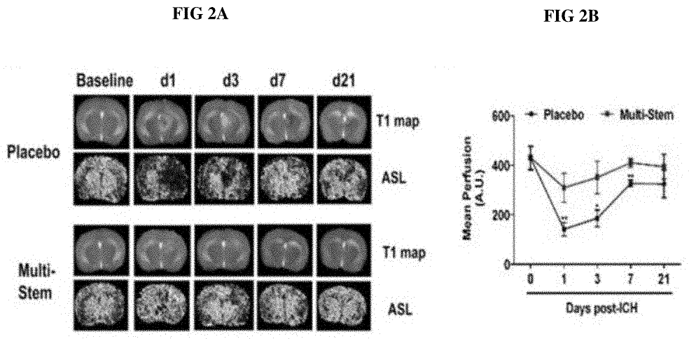

Results are shown in FIG. 2. Placebo (PBS, n=10) or MultiStem® (n=1) were intravenously administered to mice at 2 hour post-collagenase induced ICH. Cerebral perfusion was assessed by MRI (ASL; FAIR-RARE) using a 7T small animal MRI. Representative coronal brain images are provided Panel A with quantified data presented in Panel B. Data indicate that MultiStem® improves cerebral perfusion over the first week after ICH. Data are presented as mean+/−SEM and were analyzed by Student's t-test within each time point. * p<0.05, ** p<0.01 vs. placebo treated ICH mice.

### Example 5—MAPC Reduces Functional Deficit and Improves Performance After ICH

The changes observed in reduction in infarct volume and improved cerebral perfusion were mirrored by functional improvements in motor performance on grip strength test, attenuated time to cross a beam, and normalized left/right swing ratio.

Behavioral Tests

Hanging Wire Test

Grip strength was assessed by placing mice on an apparatus consisting of a 50-cm string pulled between two vertical supports. Mice were evaluated as follows: 0: falls off; 1: hangs on to string by two fore paws; 2: same as for 1 but attempts to climb on string; 3: hangs on to string by two fore paws plus one or both hind limbs; 4: hangs on to string by fore paws, vith tail wrapped around string; and 5: escapes. The highest reading of three successive trials was taken for each animal at each time point.

Narrow Beamwalk

Motor coordination was evaluated on stationary narrow beam (6 mm wide, 1 m long) over three consecutive days. The first 2 days consisted of training and performance on the beam was quantified on the third day by measuring the time required to traverse the beam. Each mouse was tested three times by a blinded investigator and the average was recorded.

Elevated Body Swing Test

Animals were held 1 cm from the base of the tail and suspended 1-5 cm above a flat surface. One swing was recorded for each suspension. A swing was defined as a >10 degree deflection from body midline or rotation about the vertical axis. Mice were placed onto the surface between suspensions, allowed to visibly reposition so that no side preferences were observed, and then resuspended. The evaluator varied the hand and standing position, and the testing area was devoid of visual cues to avoid biasing the direction of swings. 20 swings were recorded per trial, and side preference was calculated as swings to one side/total swings.

These data suggest that the acute treatment with MultiStem® following onset of hemorrhagic stroke results in profound significant improvements in locomotor and neurological benefit as evidenced across the three test administered to the animals, compared to saline only treated animals.

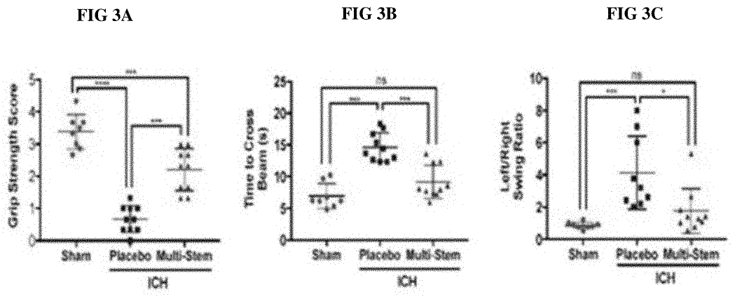

Results are shown in FIG. 3. Placebo (PBS, n=10) or MultiStem® (n=1) were intravenously administered to mice at 2 hour post-collagenase induced ICH. Neurological assessment of motor function was assessed at day 7 post-injury (or in sham-operated mice; n=8). (A) Grip strength test. (B) Narrow Beam Task. (C) Elevated body swing task. Data are mean+/−SEM and were compared using a One Way ANOVA followed by Tukey's post-hoc test. * p<0.05, **p<0.01, *** p<0.001, ns=not significant.

The outcomes described above are surprisingly good, especially in light of the fact that there is currently no approved treatment for patients suffering a hemorrhagic stroke, other than surgical evacuation in the patients where location and size of the clot lend themselves to surgery. No approved drugs or therapies exist.

The most recent pre-clinical paper evaluating experimental therapies was published in September of 2018 by the Dhandapani group, and focuses on reducing hematoma volume via inhibition of adenosine monophosphate kinase alpha-1 (AMPKal) (Vaibhav, 2018). In this paper, the same types of MRI outcomes and locomotor and neurological endpoints were evaluated as in this application.

The administration of MultiStem® cells is consistently better in hematoma reduction and blood flow outcomes, and as good if not better in locomotor outcomes, when compared to the results presented in this application. These are the best results that have been seen in studies of treatments for ICH.

The foregoing description and examples are illustrative but not exhaustive of the many aspects and embodiments encompassed by inventions herein disclosed, as will be appreciated by those skilled in the arts to which they pertain.

All publications referred to in the foregoing disclosure are incorporated into the disclosure by reference their entireties, particularly in the parts most pertinent to the subject matter for which they have been specifically cited.

1. A method of treating intracerebral hemorrhage in a subject, comprising administering to a subject in need thereof multipotent adult progenitor cells that are not embryonic stem cells, not embryonic germ cells, and not germ cells, can differentiate into at least one cell type of each of at least two of the endodermal, ectodermal, and mesodermal embryonic lineages, and are allogeneic or xenogeneic to the subject.

2. A method of treating intracerebral hemorrhage in a subject, comprising administering to a subject in need thereof multipotent adult progenitor cells that are not embryonic stem cells, not embryonic germ cells, and not germ cells, express telomerase and are allogeneic or xenogeneic to the subject.

3. A method of treating intracerebral hemorrhage in a subject, comprising administering to a subject in need thereof multipotent adult progenitor cells that are not embryonic stem cells, not embryonic germ cells, and not germ cells, are positive for oct3/4 and are allogeneic or xenogeneic to the subject.

4. A method of treating intracerebral hemorrhage in a subject, comprising administering to a subject in need thereof multipotent adult progenitor cells that are not embryonic stem cells, not embryonic germ cells, and not germ cells, undergone at least 40 cell doublings in culture prior to their use, and are allogeneic or xenogeneic to the subject.

5. A method according to claim 2, wherein can differentiate into at least one cell type of each of at least two of the endodermal, ectodermal, and mesodermal embryonic lineages.

6. A method according to claim 3, wherein can differentiate into at least one cell type of each of at least two of the endodermal, ectodermal, and mesodermal embryonic lineages.

7. A method according to claim 4, wherein can differentiate into at least one cell type of each of at least two of the endodermal, ectodermal, and mesodermal embryonic lineages.

8. A method according to claim 3, wherein said cells express telomerase.

9. A method according to claim 4, wherein said cells express telomerase.

10. A method according to claim 4, wherein said cells are positive for oct 3/4.

11. A method use according to claim 1, wherein said cells express telomerase and are positive for oct 34/.

12. A method according to claim 1, wherein said cells express telomerase and have undergone at least 40 cell doublings prior to their use.

13. A method according to claim 1, wherein said cells express oct 3/4 and have undergone at least 40 cell doublings prior to their use.

14. A method according to claim 2, wherein said cells express oct 3/4 and have undergone at least 40 cell doublings prior to their use.

15. A method according to claim 1, wherein said cells express telomerase, are positive for oct 3/4 and have undergone at least 40 cell doublings prior to their use.

16. A method according to claim 1, wherein said cells have a normal karyotype.

17. A method according to claim 1, wherein said cells are not tumorigenic.

18. A method according to claim 1, wherein said cells are not immunogenic in said subject.

19. A method according to claim 1, wherein said cells can differentiate into at least one cell type of each of the endodermal, ectodermal, and mesodermal embryonic lineages.

20. A method according to claim 1, wherein said cells are mammalian cells.

21. A method according to claim 1, wherein said cells are human cells.

22. A method according to claim 1, wherein said cells are derived from cells isolated from any one of placental tissue, umbilical cord tissue, umbilical cord blood, bone marrow, blood, spleen tissue, thymus tissue, spinal cord tissue, adipose tissue, and liver tissue.

23. A method according to claim 1, wherein said cells are derived from bone marrow.

24. A method according to claim 1, wherein the subject is a human.

25. A method according to claim 1, wherein one or more doses of 10⁴ to 10⁸ of said cells per kilogram of the subject's mass are used.

26. A method according to claim 22, wherein one or more doses of 10⁶ to 5×10⁷ of said cells per kilogram of the subject's mass are used.

27. A method according to claim 1, wherein an anti-microbial agent, an anti-fungal agent, an anti-viral agent or a combination thereof is used concurrently.

28. A method according to claim 1, wherein said cells are in a formulation comprising one or more other pharmaceutically active agents.

29. A method according to claim 1, wherein said are administered by a parenteral method, an intravenous method or a stereotactic method.

30. A method according to claim 1, wherein said cells are administered by an intravenous method.
